(19) United States
(12) Patent Application Publication
(10) Pub. No.: US 2025/0177599 A1
(43) Pub. Date: Jun. 5, 2025

(54) Nanofiltration Devise for Deactivation of Air-Filtered Pathogens on the Surface-Treated Filter Material

(71) Applicant: VÝSKUMNÝ ÚSTAV PAPIERA A CELULÓZY, A.S, Bratislava (SK)

(72) Inventors: Štefan Bohácek, Bratislava (SK); L'ubomír Sooš, Bratislava (SK); Marcela Tóth, Bratislava (SK); Monika Stankovská, Bratislava (SK); Andrej Pažitný, Bratislava (SK); Vladimír Ihnát, Bratislava (SK); Vladimír Kuna, Bratislava (SK); Jozef Balbercák, Bratislava (SK); Jirí Schwartz, Bratislava (SK)

(21) Appl. No.: 18/725,673

(22) PCT Filed: Dec. 30, 2021
(86) PCT No.: PCT/SK2021/000007

§ 371 Date: Jun. 28, 2024

(51) Int. Cl. A61L 9/20; A61L 101/26; A61L 101/30

(52) U.S. Cl. CPC A61L 9/20; A61L 2101/26; A61L 2101/30; A61L 2209/14; A61L 2209/16

(57) ABSTRACT

A nanofiltration device for inactivating droplet-transmitted pathogens. The microdroplets are captured by filtering contaminated air on a filter material/paper with the nanofibrillated cellulose and/or nanocellulose surface treatment containing antiseptic metal ions and an adjuvant in the retained residual water. The air is sucked into the openings of the nanofiltration device located at the bottom and passes into the centre of the filter cartridge, in which a germicidal emitter emits radiation in the UV-C range of the electromagnetic wavelength spectrum is mounted. Subsequently, it flows through a filter paper containing salts of antiseptic metals, from the centre of the radiator outwards through a filter paper hermetically inserted into the filter cartridge, folded harmoniously-shaped so that its surface is maximally and entirely irradiated with UV-C radiation. Antiseptic ions diffuse into the contaminated microdroplets which with UV-C radiation deactivate viruses and disinfect bacteria. Subsequently, the droplets dry out, and the deactivated viruses and bacteria are carried away by air, which, with the help of adjuvants, receive the human body and increase the population's immunity.

### TECHNICAL FIELD

The invention relates to a nanofiltration device for inactivating air-filtered pathogens transferred in a droplet manner trapped on a filter material/paper with a surface treated with a layer of nanofibrillated cellulose and/or nanocellulose containing antiseptic metal ions and adjuvants in retained residual water, in which are combined the inactivating and disinfecting effects of air filtration via mixed material with nanofibrillated cellulose and/or nanocelluloses and nitrates of silver, copper, zinc, calcium and magnesium and a germicidal emitter emitting radiation in the UV-C range of the electromagnetic wavelength spectrum. This presented device for inactivating pathogens in the air can be used in busy places with a high incidence of viruses, such as shopping centres, waiting rooms, airports, supermarkets and hypermarkets, hospitals, public transport stations and stops, schools, preschools, churches, museums, theatres, cinemas, gyms, stadiums, outdoor swimming pools, indoor swimming pools, hotels, restaurants, barracks, public transport as well as households, etc.

### BACKGROUND

Currently used devices for reducing the concentration of pathogens, which have a virucidal and bactericidal effect, are relatively readily available but do not guarantee total removal of bacteria or complete inactivation of viruses located on the surface of dust particles and inside droplets contained in the air. In addition, they are mainly produced specifically for the medical industry. At the same time, they are also commonly applied in industrial facilities integrated or integrated into hospital and medical buildings with the provision of medical care in their rooms such as entrance areas, waiting rooms, ambulances, hospital halls, and also other hospital and outpatient rooms with a high risk of infection, or with high demands on the cleanliness of the environment.

The devices are also valuable for the manufacture of susceptible to any pathogens present in the air, for example, in the manufacture of pharmaceuticals or pharmaceutical precursors and biochemical and other manufacturing. Commercially available devices are inefficient in inactivating pathogens in the air or do not contain an activated surface on which would inactivate pathogens. Pathogens include pathogenic viruses, microscopic fungal spores, fungi, fungal spores and bacteria, which are found in the air on microparticles in the form of biological aerosols—bioaerosols. Since many of the viruses and bacteria found in bioaerosols are still partially active, respectively live, can continue to replicate and multiply on suitable material in the form of droplets and dust particles. In addition to these microparticles, bioaerosols also contain other particles from living organisms such as pollen grains, plant seeds, wood dust particles, animal hair fragments, animal exudates, insects, insect secretions and other biological material that can act as a breeding ground for harmful pathogens. Said particles can be present in the air either separately, where they are active and survive only for a short time, or they are present on a suitable carrier in the form of dust particles and droplets causing the so-called droplet infection. Droplet infection is the spread of viruses (rhinitis, influenza, including so-called swine and avian, coronavirus, etc.) and bacteria (e. g. tuberculosis) in the “fog” of contaminated droplets of mucus and saliva in the air. Another example is the disease Legionellosis, the causative agent of which, the bacterium Legionella pneumophila, spreads in bioaerosol from humidifiers and air conditioning systems (X. Gong et al.: Molecular typing of Legionella pneumophila from air-conditioning cooling waters using mip gene, SBT, and FAFLP methods Journal of Microbiological Methods 139 (2017) 1-7pp). It is, therefore, necessary to consider the use or the intensity of the use of air conditioning equipment or include effective devices for the inactivation of harmful pathogens at the outlets of air-conditioning units and air-conditioning units.

The high affinity of several polar compounds such as ammonia, water, hydrogen sulphide and phosphane in the gas or liquid phase for cellulose, the fibres of which have a negatively charged surface, is known in the professional literature (A. Onur et al.: Cellulose fibre-perlite depth filters with cellulose nanofibre top coating for improved filtration performance. Colloids and Surfaces A: Physicochemical and Engineering Aspects 583 (2019) 123997). The most common carrier of pathogens in the air among these compounds is water, which is, therefore, the essential part of bioaerosols. Due to the water, but also due to the mentioned polar compounds present in the polluted air, harmful bacteria and viruses survive in the air longer, and the duration of exposure then affects the infection of a more significant part of the population in places with higher human concentrations such as hospitals, outpatient areas and quarantine stations.

The negatively charged surface of the cellulose is attracted to the positively charged particles, while in the case of water molecules and polar compounds bearing a hydrogen atom, they are hydrogen atoms with a positive fractional charge; therefore, the water molecules and possible polar compounds will be oriented inside the cellulose film surface and the oxygen atom with a negative fractional charge outwards. Additional water molecules and optional polar compounds will subsequently be arranged in a similar orientation, as the oxygen atom contains two free electron pairs, or optionally another electronegative element may also contain free electron pairs, which form additional hydrogen bonds. Some bioengineering techniques also use methods to treat nanocellulose from native fibrous networks of living organisms while modifying specific properties such as porosity (Ashrafi et al.: Bioengineering tunable porosity in bacterial nanocellulose matrices. Soft Matter 15 (45) (2019) 9359-9367). Membranes obtained from the nanocellulose thus prepared can also be used as a mechanical and physicochemical barrier for pathogen-containing microparticles.

The microorganisms are released into the air by aqueous dispersion from their growth site or colonized surfaces. Bioaerosols can contribute to the spread of infectious diseases, as well as emerging droplet infections such as SARS (Severe Acute Respiratory Syndrome), acute intoxications, allergies, asthma and the spread of precancerous disorders. To disinfect and sterilize the air, thus to inactivate the above-mentioned pathogenic microorganisms, various ozone-based devices are used (J. Arlemark (SE): WO 2013/110782 A1). The devices using UV-A group of ultraviolet light with a wavelength range from 400 nm to 315 nm are also known used for air disinfection and deodorization (SJ Palackal (IN) et al.: WO 2019/073474 A1). A device can perform the process of disinfecting or inactivating pathogenic particles called a UV-C sterilizer according to a patent for an invention granted to Deal and published in 2013 (J L Deal (US): UV-C sterilizer. EP 2 174 670 B1), which disinfects or inactivates pathogenic particles using UV-C radiation, but said device does not provide for another possibility of their inactivation. The number of microparticles containing harmful pathogens, resp. pathogenic particles also depend on the relative humidity, which is affected by the partial pressure of water vapour in the air its temperature and pressure. The relative humidity depends on the water vapour content in the air stream (A. Pažitný (SK), Š. Boháček (SK), P. Medo (SK), Z. Brezániová (SK), A. Russ (SK), M. Stankovská (SK), V. Ihnát (SK), J. Schwartz (SK), J. Gigac (SK), J. Balberčák (SK): Spiral-based recuperation device with U-tube condensate drain integration SK 50043-2016 A3).

Another way to detoxify the water in the air is to add biocidal additives to a carrier that comes into contact with the pathogen or droplet containing the pathogen and then adsorbs on it. However, the methods have been developed mainly for water in a compact state, i.e. for polluted and biologically harmful water in the liquid form, not for application to water droplets in the air. Ion-containing compositions such as ions of calcium, iron, carbonates, chlorides, nitrates, phosphates and sulphates are used as biocides (NA Samad (US), DD Back (US): Water disinfection method using metal-ligand complexes. U.S. Pat. No. 5,632,904 A). However, in the presence of sulphane in the air, silver or copper salts can be used (P. R. Warburton (US), R. S. Sawtelle (US): Filter for gas sensor. U.S. Pat. No. 6,284,545 B1). Aqueous solutions of silver and copper salts have a biocidal effect. Several fungicidal compositions containing copper, magnesium, zinc or their salts are also known and tested since 1986 (Y. Honma (JP), Y. Arimoto (JP), A. Misato (JP), T. Toriyama (JP), K. Tomono (JP); Fungicide composition for agricultural and horticultural use (JP S61 233 606 A). Several breaths of air and woven material cleaners also contain HEPA filters (“High-Efficiency Particulate Air” filters), made of glass microfibers and folded in multiple folds. HEPA filters are ranked in the cleaning equipment as the last filters in the order from the mouth of the device that traps dust. The task of HEPA filters is to capture even the finest particles, such as dust, pollen, cigarette smoke, mite or mould faeces, which were not caught by the paper bag or the filters in front of it. However, HEPA filters can be combined with various other filters, and a filter layer mounted in a cleaning device formed by HEPA filters can be placed in front of or behind another activated filter material. The filter layers of HEPA filters can be combined with various filter layers containing organic or inorganic materials, such as layers containing activated carbon or silver cation nanoparticles, as published in 2016 (M. Gu (CN): Remotely controllable air purification water maker, CN 105735416 A). The addition of different active filter layers significantly increases the filtration effect of HEPA filters.

### SUMMARY

The essence of the invention lies in its ability to capture, disinfect and inactivate pathogenic microorganisms and their residues, which are present in polluted air and injected with droplets in the air and dust from the surrounding space into the system of openings at the entrance to the nanofiltration device. The orifice system of the nanofiltration device is located at the bottom part of the device into which air is sucked, which passes into the space in the middle of the filter cartridge, in which a germicidal emitter emitting radiation in the UV-C range of the electromagnetic wavelength spectrum is mounted. The air thus irradiated flows through a filter sheet containing antiseptic metal salts, from the centre of the emitter outwards through a combined filter material hermetically inserted into the filter cartridge, accordion-shaped folded so that its surface is and the entire surface is irradiated with UV-C radiation. On the surface of the specially treated filter paper, adhesive droplets, hydrophilic forces, and hydrogen bonds trap microdroplets of saliva infected by droplet-transmitted enteroviruses and bacteria, which are permanently irradiated with UV-C radiation, while Fick's law of diffusion, which ensures the transfer of antiseptic metal cations and adjuvants due to the concentration gradient into microdroplets, where together with the action of UV-C radiation they inactivate viruses and kill the bacteria present. On this principle, the nanofiltration device causes the inactivation of air-filtered pathogens, mainly through inorganic additives in the retained residual water. The purified air then flows through the filter paper into the space between the filter cartridge and the outer wall of the device and is directed upwards by the fan (10), where it is then discharged at the height of the airways of an average person. After the optimal time of action of antiseptic effects of metal cations, salt anions and UV-C radiation due to the combination of radiation and airflow, droplets dry and airborne inactivated viruses and dead bacteria, which subsequently get into the human body with adjuvants and increase population immunity.

The filter sheet of the nanofiltration device contains nanofibrillated cellulose and/or nanocellulose and adjuvants and nitrates of silver, copper, zinc, calcium, and magnesium. Inactivation of pathogens is carried out by the combined effect of the described filter paper and a germicidal emitter emitting radiation in the UV-C range of the electromagnetic wavelength spectrum. The sheet with a specific content of nanofibrillated cellulose and/or nanocellulose and nitrates of silver, copper, zinc, calcium and magnesium forms a mechanical and physicochemical barrier for microdroplets in the air stream containing pathogens. Germicidal emitter, located in the middle of the nanofiltration device, emitting radiation in the UV-C electromagnetic spectrum of wavelengths in the range preferably from 280.0 nm to 250.0 nm (in extreme cases up to 210.0 nm), where harmful ozone is not yet formed, and the device can be can also be used in the presence of humans, animals and plants.

The process of inactivation of pathogenic particles by UV-C radiation is intensified compared to previous industrial solutions in this nanofiltration device by applying nanofibrillated cellulose and/or nanocellulose with residual water containing antiseptic inorganic salts. The addition of nanofibrillated cellulose and/or nanocellulose optimizes the size and distribution of the pores and increases the hydrophilic and antiseptic effects of the surface due to the negative charge on the surface. Chemically and physically, it is a sheet based on nanofibrillated cellulose and/or nanocellulose with a pore content in the range from 10 nm to 25 μm, with a specific pore surface in the range from 20 cm²·g⁻¹ to 75 m²·g⁻¹ and a specific a pore volume in the range from 0.05 cm³·g⁻¹ to 0.70 cm³·g⁻¹, the pore surface with the stated ranges of given parameters having the ability to adsorb microparticles containing harmful or partially pathogens. The sheet also contains an added admixture of nitrate salts (AgNO₃, Cu(NO₃)₂, Zn(NO₃)₂, Ca(NO₃)₂, and Mg(NO₃)₂) in a concentration below their solubility limit. The number of microscopic particles containing harmful pathogens adsorbed on nanofibrillated cellulose and/or nanocellulose fibres containing admixtures of said inorganic salts also depends on the relative humidity in the air stream as the microscopic particles usually accumulate on dust particles and are absorbed by water microdroplets. The accumulation, respectively adsorption of these water-containing particles is highly efficient, especially in the case of the application of paper sheets with admixtures of said nitrate salts, which increase the germicidal effect of radiation in the UV-C electromagnetic spectrum emitted by the germicidal emitter. Air stream passes through the nanofiltration device through openings in the lower part of the nanofiltration device, into the space in which the germicidal emitter is longitudinally placed in the middle opens, and the air stream flows around it. Subsequently, the air stream passes through a sheet with a surface treated with a layer of nanofibrillated cellulose and/or nanocellulose and nitrates of silver, copper, zinc, calcium and magnesium. The microparticles are partially disinfected during the flow through the forced air space. The germicidal emitter is centrally located by the action of ultraviolet radiation of the electromagnetic wavelength range ranging from 280.0 nm to 210.0 nm, which is emitted by this germicidal emitter. A fan provides the forced airflow in the nanofiltration device (10) mounted above the filtered battery in the outlet tapered part of the device or at the inlet to the battery—if necessary to increase the air circulation speed. The flow of intake contaminated air is guided by the action of the fan (s) to a surface treated sheet with a layer of nanofibrillated cellulose and/or nanocellulose and nitrates of silver, copper, zinc, calcium and magnesium or their various combinations by omitting one or more of them to achieve a required share of price and effect. The fibres of this material have the ability to adsorb on their surface living, respectively biologically active pathogenic microparticles and residual bioaerosols containing pathogenic microorganisms, or parts thereof which have not been inactivated, are additionally inactivated on nanofibrillated cellulose and/or nanocellulose fibres, the biocidal agent being present nitrate salts, which diffuse into microdroplets to pathogens. The sheet, folded accordion-shaped and rolled into a cylindrical shape, is hermetically inserted into the filter cartridge so that the active surface is maximized and irradiated with UV-C radiation and the entire air stream passes through this active filter material.

Nanofiltration device for inactivation of air-filtered pathogens on combined filter material/filter paper with surface treatment—a layer of nanofibrillated cellulose-containing antiseptic metal ions and adjuvants in retained residual water, which combines air filtration using filter paper with the addition of nanofibrillated/nanocelluloses and nitrates of silver, copper, zinc, calcium and magnesium and a germicidal emitter emitting can achieve radiation in the UV-C range of the electromagnetic wavelength spectrum, an inactivation efficiency of pathogens of more than 99.99%. Compared to conventional airborne pathogen inactivation devices, this results in much higher pathogen inactivation efficiencies, as various combination devices are often used in medical and household buildings or in buildings where air disinfection is required without the use of special sheets with by the addition of nanofibrillated cellulose and/or nanocellulose and nitrates of silver, copper, zinc, calcium and magnesium, with a pathogen inactivation efficiency ranging from 85% to 90%.

The volume and position of the relevant space in which the germicidal emitter is located in the UV-C range of the electromagnetic wavelength spectrum are adapted so that the airflow rate inside the pores in the sheet is achievable at a level in the range from 0.01 m·s⁻¹ to 7.9 m·s⁻¹. The airflow is directed through the active walls of the filter material. It exits through the outlet openings of this device upwards or to the upper opening of the device, which is optimally at the height of the breathing openings of an average person.

### BRIEF DESCRIPTION OF THE DRAWINGS

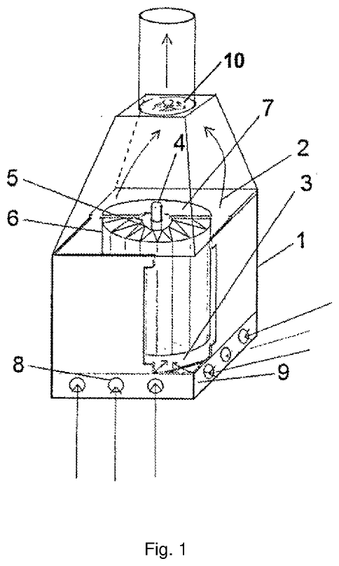

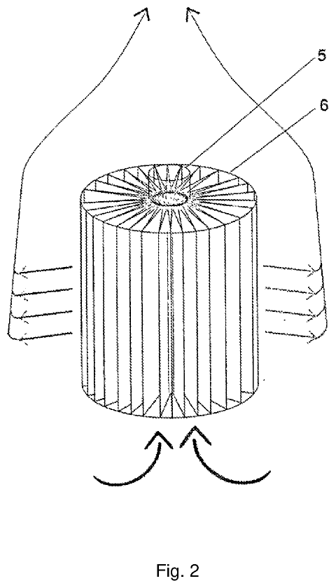

The invention is elucidated using two drawings, in which FIG. 1 shows the arrangement and position of the space concerning a nanofiltration device for deactivating air-filtered pathogens on a combined surface-treated filter material/filter paper with a layer of nanofibrillated cellulose and/or nanocellulose containing antiseptic metal ions and adjuvants in a retained water residue, with germicide emitter emitting ultraviolet radiation in the UV-C range of wavelengths of electromagnetic radiation and airflow in a nanofiltration device, and FIG. 2 is a surface-treated filter material/filter paper sheet with a nanofibrillated cellulose and/or nanocellulose containing nitrate salt additives on the inner side of the cartridge and air flowing from the filter paper sheet towards the upper opening of the device.

### DETAILED DESCRIPTION

### Example 1

Nanofiltration device 1 for inactivating air-filtered pathogens on surface-treated filter material/paper with inorganic additives and nanofibrillated cellulose and/or nanocellulose connected to the surrounding environment through the upper opening 2 according to FIG. 1 has a space 3 formed in the inner part, in which a germicidal emitter 4 in the shape of a cylinder or a U-tube is mounted in a filter cartridge with a accordion-shaped folded filter sheet. The germicidal emitter 4 is located axially, is parallel to the direction of the incoming airflow to the filter cartridge and is longitudinally located in the central part of the interior of the nanofiltration device and emits radiation doses ranging from 400 μW to 500 W with exposure values ranging from 2 seconds to 7200 seconds. The germicidal electromagnetic radiation from the emitter has wavelengths ranging from 280.0 nm to 210.0 nm. The peripheral walls of the interior of the nanofiltration device 1 are bounded by a long-term stable material in the UV-C range of the electromagnetic wavelength spectrum and may be in the shape of a prism or a vertically positioned cylinder terminated by a conical constriction and a discharge tube, preferably with an adjustable outlet height. In the middle part of the inner space 3 there is an accordion-shaped folded filter sheet 5, which is hermetically placed in the filter cartridge 6. According to FIG. 2, the filter cartridge is hermetically sealed in the upper part by a cover 7 so that all intake air is filtered through an accordion-shaped folded filter sheet. The filter paper sheet 5 for air filtration is surface-treated with a material of nanofibrillated cellulose and/or nanocellulose with a pore content from 10.0 nm in size, with a specific pore surface area of 20 m²·g⁻¹ and a specific pore volume of 0.10 cm³·g⁻¹. The sheet also contains an added admixture of nitrate salts (AgNO₃, Cu(NO₃)₂, Zn(NO₃)₂, Ca(NO₃)₂ and Mg(NO₃)₂) with a total charge of 5.0 g per 1 m² sheet and an adjuvants with a total charge in the range from 0.10 to 0.59 g per 1 m² sheet. The molar ratio of the individual nitrate salts AgNO₃:Cu(NO₃)₂:Zn(NO₃)₂:Ca(NO₃)₂:Mg(NO₃)₂ is 1:1:1:1:1. The pore surface of the sheet with the stated parameters is able to adsorb microparticles containing pathogenic microorganisms or parts thereof.

The nanofiltration device according to FIG. 1 works as follows. Polluted air containing pathogenic particles containing viruses, spores of microscopic fungi, moulds, fungal spores, bacteria, algae, as well as residues of pathogenic microorganisms flowing into the nanofiltration device 1 is sucked through the system of holes 8, flows through the pedestal 9, from which it is distributed to the space 3, in which the germicidal emitter 4 is longitudinally mounted. During the airflow, the germicidal emitter 4 emits radiation doses ranging from 400 μW to 200 W with exposure values ranging from 2 seconds to 7200 seconds, depending on the types of pathogenic particles in the flowing air present. The radiation-treated air in the UV-C radiation range of the electromagnetic wavelength spectrum forcibly flows to the upper part, from where it flows to the filter paper sheet 5 in the middle of the filter cartridge. Subsequently, the air disinfected in this way is discharged from the nanofiltration device 1 through an opening 2, which is located in the upper part of the nanofiltration device 1. The air is sucked out of the nanofiltration device 1 by a fan 10.

The nanofiltration device 1 can be used in all cases where biological aerosols containing viruses, microscopic spores, fungi, fungal spores, bacteria and algae, as well as residues of pathogenic microorganisms, which are harmful to humans accumulate. It is mainly used in buildings of medical facilities and households or in other buildings where air disinfection and residual pathogens are removed.

### Example 2

Nanofiltration device 1 for inactivation of air-filtered pathogens by using inorganic additives on a cellulose carrier according to Example 1, using a filter paper sheet 5 without the addition of AgNO₃ with a ratio of nitrate salts Cu(NO₃)₂:Zn(NO₃)₂:Ca(NO₃)₂:Mg(NO₃)₂ in the range from 1:1:1:1 to 2:1:1:1, depending on the content of nanofibrillated cellulose and/or nanocellulose and adjuvants in the filter paper sheet 5.

### Example 3

Nanofiltration device 1 for inactivation of air-filtered pathogens by using inorganic additives on a cellulose carrier according to Example 1, using a filter paper sheet 5 without the addition of Cu(NO₃)₂: with a ratio of nitrate salts AgNO₃:Zn(NO₃)₂:Ca(NO₃)₂:Mg(NO₃)₂ in the range from 1:1:1:1 to 2:1:1:1 depending on the content of nanofibrillated cellulose and/or nanocellulose and adjuvants in the filter paper sheet 5.

### Example 4

Nanofiltration device 1 for inactivation of air-filtered pathogens by using inorganic additives on a cellulose carrier according to Example 1, using a filter paper sheet 5 without the addition of AgNO₃ and Cu(NO₃)₂ with a ratio of nitrate salts: Zn(NO₃)₂:Ca(NO₃)₂:Mg(NO₃)₂ in the range from 1:1:1 to 2:1:1, depending on the content of nanofibrillated cellulose and/or nanocellulose and adjuvants in the filter paper sheet 5.

### Example 5

Nanofiltration device 1 for inactivation of air-filtered pathogens by using inorganic additives on a cellulose carrier according to Example 1, using a filter paper sheet 5 without the addition of AgNO₃, Cu(NO₃)₂ and Ca(NO₃)₂ with a ratio of nitrate salts: Zn(NO₃)₂:Mg(NO₃)₂ in the range from 1:1 to 2:1 depending on the content of nanofibrillated cellulose and/or nanocellulose and adjuvants in the filter paper sheet 5.

### Example 6

Nanofiltration device 1 for inactivation of air-filtered pathogens by using inorganic additives on a cellulose carrier according to Example 1, using a filter paper sheet 5 only with the addition of AgNO₃ with applicated amount of 0.1 to 5.0 g·m⁻², depending on the content of nanofibrillated cellulose and/or nanocellulose and adjuvants in the filter paper sheet 5 and the nature of the common infection.

### Example 7

Nanofiltration device 1 for inactivation of air-filtered pathogens by using inorganic additives on a cellulose carrier according to Example 1, using a filter paper sheet 5 only with the addition of Cu(NO₃)₂ with applicated amount of 0.1 to 15.0 g·m⁻² depending on the content of nanofibrillated cellulose and/or nanocellulose and adjuvants in the filter paper sheet 5 and the nature of the common infection.

### Example 8

Nanofiltration device 1 for inactivation of air-filtered pathogens by using inorganic additives on a cellulose carrier according to Example 1, using a filter paper sheet 5 only with the addition of Zn(NO₃)₂ with applicated amount 0.1 to 15.0 g·m⁻² depending on the content of nanofibrillated cellulose and/or nanocellulose and adjuvants in the filter paper sheet 5 and the nature of the common infection.

### Example 9

Nanofiltration device 1 for inactivation of air-filtered pathogens by using inorganic additives on a cellulose carrier according to Example 1, using a filter paper sheet 5 only with the addition of Ca(NO₃)₂ with applicated amount 0.1 to 15.0 g·m⁻² depending on the content of nanofibrillated cellulose and/or nanocellulose and adjuvants in the filter paper sheet 5 and the nature of the common infection.

### Example 10

Nanofiltration device 1 for inactivation of air-filtered pathogens by using inorganic additives on a cellulose carrier according to Example 1, using a filter paper sheet 5 only with the addition of Mg(NO₃)₂ with applicated amount 0.1 to 15.0 g·m⁻² depending on the content of nanofibrillated cellulose and/or nanocellulose and adjuvants in the filter paper sheet 5 and the nature of the common infection.

### Example 11

Nanofiltration device 1 for inactivation of air-filtered pathogens by using inorganic additives on a cellulose carrier according to Example 1, using a filter paper sheet 5 in the middle of the filtration cartridge 6 at a rate in the range from 0.05 m·s⁻¹ to 7.9 m·s⁻¹, the air temperature is in the range from 11.3° C. to 27.9° C. and its relative humidity is in the range from 10% to 99%.

This work was supported by the Slovak Research and Development Agency under the Contract no. PP-COVID-20-0103.

### INDUSTRIAL APPLICABILITY

The invention's applicability is in the field of the chemical industry, food industry, pharmaceutical industry, electrical industry, medical equipment industry, biotechnology industry and various other industrial areas. Also in microbiology, aeromicrobiology, especially at the places with the need for additional removal of residual pathogens and in all busy places with a high incidence of viruses such as shopping centres, waiting rooms, airports, supermarkets and hypermarkets, hospitals, public transport stations and stops, schools, pre-schools, churches, museums, theatres, cinemas, gyms, stadiums, outdoor swimming pools, indoor swimming pools, hotels, restaurants, barracks, etc., in public transport as well as in households.

By applying the invention, it is possible to achieve the inactivation efficiency of pathogens containing viruses, microscopic spores, fungi, mould spores, bacteria, algae and residues of pathogenic microorganisms in the form of bioaerosols which are harmful to humans, at optimal airflow settings in pathogen inactivation devices 99.99%.

1. A nanofiltration device of cylindrical or prismatic shape for inactivation of air-filtered pathogens through a filter containing inorganic additives in residual water on a cellulosic carrier with a flat base connected to the environment via a perforated base/pedestal with an interior space bounded by long-term stable material against the action of radiation in the UV-C range of the electromagnetic wavelength spectrum, placed longitudinally in the interior space of this device into the shape of a cylinder, longitudinally located in the interior space of this device into the shape of a cylinder, in which middle is longitudinally (axially) a germicidal emitter in the form of a cylinder or a U tube, emitting UV-C radiation, placed parallel to the direction of the incoming airflow into the interior of the filter cartridge hermetically insulated in the lower part of the interior space so that the air sucked in from the environment through the openings in the lower part of the device reaches only the centre of the cartridge, which is hermetically sealed from the top, so the airflow is directed through accordion-shaped arranged porous walls of filter paper sheet, wherein it contains nanofibrillated cellulose from corn-based distillery refuse and/or nanocellulose, adjuvants—aluminum compounds AlPO₄, Al(OH)₃, (Al)₂PO₄SO₄OH and chitosan added below their solubility limit and nitrate salts Cu(NO₃)₂, Zn(NO₃)₂, Ca(NO₃)₂ and Mg(NO₃)₂ or other soluble salts of Cu, Zn, Ca and Mg in concentration below their solubility limit and the filter paper sheet is folded accordion-shaped in the filter cartridge so that its surface is maximal and completely irradiated with UV-C radiation and fan/fans is/are installed below and/or above the filter cartridge to provide a forced flow of air through the pores of the accordion-shaped folded filter sheet.

2. The nanofiltration device for inactivation pathogens filtered from the air by means of inorganic additives on a cellulose carrier with the flat base connected with the surrounding environment via a base/pedestal according to claim 1, wherein it contains nanofibrillated cellulose and/or nanocellulose, adjuvants and nitrate salts, Zn(NO₃)₂, Ca(NO₃)₂ and Mg(NO₃)₂ or other soluble salts of Zn, Ca and Mg in concentrations below their solubility limit and the filter material is folded accordion-shaped in the filter cartridge so that its surface is maximal and completely irradiated with UV-C radiation and fan/fans is/are installed below and/or above the filter cartridge to provide a forced flow of air through the pores of the accordion-shaped folded filter sheet.

3. The nanofiltration device for inactivation pathogens filtered from the air by means of inorganic additives on a cellulose carrier with the flat base connected with the surrounding environment via a base/pedestal according to claim 1, wherein it contains nanofibrillated cellulose and/or nanocellulose, adjuvants and nitrate salts Cu(NO₃)₂, Ca(NO₃)₂ and Mg(NO₃)₂ or other soluble salts of Cu, Ca and Mg in concentrations below their solubility limit together with filter material which is folded accordion-shaped in the filter cartridge so that its surface is maximal and completely irradiated with UV-C radiation, and fan/fans is/are installed below and/or above the filter cartridge to provide a forced flow of air through the pores of the accordion-shaped folded filter sheet.

4. The nanofiltration device for inactivation pathogens filtered from the air by means of inorganic additives on a cellulose carrier with the flat base connected with the surrounding environment via a base/pedestal according to claim 1, wherein it contains nanofibrillated cellulose and/or nanocellulose, adjuvants and nitrate salts Cu(NO₃)₂, Zn(NO₃)₂, and Ca(NO₃)₂ or other soluble salts of Cu, Zn, and Ca in concentrations below their solubility limit together with filter material which is folded accordion-shaped in the filter cartridge so that its surface is maximal and completely irradiated with UV-C radiation, and fan/fans is/are installed below and/or above the filter cartridge to provide a forced flow of air through the pores of the accordion-shaped folded filter sheet.

5. The nanofiltration device for inactivation pathogens filtered from the air by means of inorganic additives on a cellulose carrier with the flat base connected with the surrounding environment via a base/pedestal according to claim 1, wherein it contains nanofibrillated cellulose and/or nanocellulose, adjuvants and nitrate salts Cu(NO₃)₂, Zn(NO₃)₂ and Mg(NO₃)₂ or other soluble salts of Cu, Zn and Mg in concentrations below their solubility limit together with the filter material which is folded accordion-shaped in the filter cartridge so that its surface is maximal and completely irradiated with UV-C radiation, and fan/fans is/are installed below and/or above the filter cartridge to provide a forced flow of air through the pores of the accordion-shaped folded filter sheet.

6. The nanofiltration device for inactivation pathogens filtered from the air by means of inorganic additives on a cellulose carrier with the flat base connected with the surrounding environment via a base/pedestal according to claim 1, wherein it contains nanofibrillated cellulose and/or nanocellulose, adjuvants and nitrate salts Cu(NO₃)₂, Zn(NO₃)₂ or other soluble salts of Cu and Zn in concentrations below their solubility limit. The filter material is folded accordion-shaped in the filter cartridge so that its surface is maximal and completely irradiated with UV-C radiation, and fan/fans is/are installed below and/or above the filter cartridge to provide a forced flow of air through the pores of the accordion-shaped folded filter sheet.

7. The nanofiltration device for inactivation pathogens filtered from the air by means of inorganic additives on a cellulose carrier with the flat base connected with the surrounding environment via a base/pedestal according to claim 1, wherein it contains nanofibrillated cellulose and/or nanocellulose, adjuvants and nitrate salts Cu(NO₃)₂, Ca(NO₃)₂ or other soluble salts of Cu and Ca in concentrations below their solubility limit and the filter material is folded accordion-shaped in the filter cartridge so that its surface is maximal and completely irradiated with UV-C radiation and fan/fans is/are installed below and/or above the filter cartridge to provide a forced flow of air through the pores of the accordion-shaped folded filter sheet.

8. The nanofiltration device for inactivation pathogens filtered from the air by means of inorganic additives on a cellulose carrier with the flat base connected with the surrounding environment via a base/pedestal according to claim 1, wherein contains nanofibrillated cellulose and/or nanocellulose, adjuvants and nitrate salts Cu(NO₃)₂, Mg(NO₃)₂ or other soluble salts of Cu and Mg in concentrations below their solubility limit and the filter material is folded accordion-shaped in the filter cartridge so that its surface is maximal and completely irradiated with UV-C radiation and fan/fans is/are installed below and/or above the filter cartridge to provide a forced flow of air through the pores of the accordion-shaped folded filter sheet.

9. The nanofiltration device for inactivation pathogens filtered from the air by means of inorganic additives on a cellulose carrier with the flat base connected with the surrounding environment via a base/pedestal according to claim 1, wherein it contains nanofibrillated cellulose and/or nanocellulose, adjuvants and nitrate salts Ca(NO₃)₂, Zn(NO₃)₂ or other soluble salts of Ca and Zn in concentrations below their solubility limit and the filter material is folded accordion-shaped in the filter cartridge so that its surface is maximal and completely irradiated with UV-C radiation and fan/fans is/are installed below and/or above the filter cartridge to provide a forced flow of air through the pores of the accordion-shaped folded filter sheet.

10. The nanofiltration device for inactivation pathogens filtered from the air by means of inorganic additives on a cellulose carrier with the flat base connected with the surrounding environment via a base/pedestal according to claim 1, wherein filtrated sheet it contains nanofibrillated cellulose and/or nanocellulose, adjuvants nitrate salts Mg(NO₃)₂, Zn(NO₃)₂ or other soluble salts of Mg and Zn in concentrations below their solubility limit and the filter material is folded accordion-shaped in the filter cartridge so that its surface is maximal and completely irradiated with UV-C radiation and fan/fans is/are installed below and/or above the filter cartridge to provide a forced flow of air through the pores of the accordion-shaped folded filter sheet.
